(19) United States
(12) Patent Application Publication
(10) Pub. No.: US 2024/0124509 A1
(43) Pub. Date: Apr. 18, 2024

(54) SEPARATION OF CHARGED OLIGOSACCHARIDES

(71) Applicant: GLYCOM A/S, Hørsholm (DK)

(72) Inventor: Martin MATWIEJUK, Hamburg (DE)

(21) Appl. No.: 18/257,979

(22) PCT Filed: Dec. 17, 2021
(86) PCT No.: PCT/IB2021/061932

§ 371 Date: Jun. 16, 2023

(30) Foreign Application Priority Data
Dec. 18, 2020 (DK) PA 2020 01430

(51) Int. Cl. C07H 7/033; B01D 15/36; B01J 41/07; C07H 1/06

(52) U.S. Cl. CPC C07H 7/033; B01D 15/363; B01J 41/07; C07H 1/06

(57) ABSTRACT

The present invention concerns a method for separating different oligosaccharides having at least one carboxylic acid group by contacting them with basic anion exchange resins.

### FIELD OF THE INVENTION

The present invention concerns a method for separating different oligosaccharides having at least one carboxylic acid group, also referred to as charged oligosaccharides. The method allows for the high throughput separation of oligosaccharides that are otherwise difficult to separate in non-chromatographic methods and involves the use of a weakly basic macroporous anion exchange resin.

### BACKGROUND OF THE INVENTION

Oligosaccharides, such as human milk oligosaccharides (HMOs), may be prepared by various different methods. These methods typically include fermentation of a bacterial host, including downstream processing of the fermentation broth. Such fermentation methods work well for smaller and less complex oligosaccharides, such as 3′-sialyllactose (3′-SL) and 6′-sialyllactose (6′-SL), but not as well for larger and more complex oligosaccharides. This is particular true for charged oligosaccharides, i.e. oligosaccharides containing at least one carboxylic acid group.

In order to prepare larger and more complex oligosaccharides, trans-glycosidase reactions have been employed where a monosaccharide unit is transferred by enzymatic catalysis from a donor to an acceptor oligosaccharide. One such example is the transfer of a sialic acid unit from a donor, such as 3′-SL or 6′-SL, to an acceptor, such as 3-FL, LNT or LNnT, by using a trans-sialidase enzyme (see e.g. WO 2016/157108, WO 2016/199071). However, these reactions result in an equilibrium between the starting educts and the oligosaccharide product. In the case of charged oligosaccharides, the donor and the product are not easily separated by the methods known in the art because they both contain at least one carboxylic acid group. The methods currently available are low-throughput methods, such as gel chromatographic methods.

Accordingly, there is a need in the art for a method that allows for high-throughput separation of charged oligosaccharides.

### SUMMARY OF THE INVENTION

In one aspect, the present invention concerns a method of separating a first oligosaccharide containing at least one carboxylic acid group from a mixture comprising at least said first oligosaccharide and a second oligosaccharide containing at least one carboxylic acid group, wherein said first oligosaccharide contains at least one monosaccharide unit less than the second oligosaccharide, comprising the steps of:
a) providing said mixture in a solvent with a pH level to ensure that at least 90% of the carboxylic acid groups of the first and the second oligosaccharides exist in protonated (free acid) form, and
b) applying the mixture on or contacting the mixture with a weakly basic macroporous anion exchange resin.

In one embodiment, the method further comprises step c) following step b), being applying the eluate or filtrate from step b) on or contacting said eluate or filtrate with a basic anion exchange resin, such as a weakly basic anion exchange resin of the gel type.

The method of the invention is efficient in separating the oligosaccharides containing carboxylic acid groups from each other so that the first oligosaccharide binds to the weakly basic macroporous anion exchange resin whereas the second oligosaccharide does substantially not, and thus provides high levels of purity of the larger oligosaccharides.

In other aspect, the present invention relates to a method of separating a second oligosaccharide containing at least one sialyl group from a mixture comprising a first oligosaccharide containing at least one sialyl group, said first oligosaccharide and optionally a neutral oligosaccharide, wherein said first oligosaccharide contains at least one monosaccharide unit less than the second oligosaccharide, comprising the steps of:
a) providing said mixture in a solvent with a pH level to ensure that at least 90% of the sialyl groups of the first and the second oligosaccharides exist in protonated (free acid) form,
b) applying the mixture on or contacting the mixture with a weakly basic macroporous anion exchange resin, ensuring the binding of the first oligosaccharide to the resin,
c) applying the eluate of step b) on or contacting the eluate of step b) with a basic anion exchange resin, such as a weakly basic anion exchange resin of the gel type, ensuring the binding of the second oligosaccharide to the resin,
d) eluting the second oligosaccharide from the resin, and
e) isolating the second oligosaccharide from the eluate of step d).

### DETAILED DESCRIPTION OF THE INVENTION

The present invention concerns a method of separating a first oligosaccharide containing at least one carboxylic acid group from a mixture comprising at least said first oligosaccharide and a second oligosaccharide containing at least one carboxylic acid group, wherein said first oligosaccharide contains at least one monosaccharide unit less than the second oligosaccharide, comprising the steps of:
a) providing said mixture in a solvent with a pH level to ensure that at least 90% of the carboxylic acid groups exist in protonated (free acid) form,
b) applying the mixture obtained in step a) on or contacting the mixture obtained in step a) with a weakly basic macroporous anion exchange resin.

Step b) of the method of invention ensures that more first oligosaccharide binds to the resin than second oligosaccharide and the second oligosaccharide is accumulated in the liquid (mobile) phase, therefore the separation of the first and the second oligosaccharide from each other is possible.

In one embodiment, the present invention concerns a method of separating a first oligosaccharide containing at least one carboxylic acid group from a mixture comprising at least said first oligosaccharide and a second oligosaccharide containing at least one carboxylic acid group, wherein said first oligosaccharide contains at least one monosaccharide unit less than the second oligosaccharide, comprising the steps of:
a) providing said mixture in a solvent with a pH level to ensure that at least 90% of the carboxylic acid groups exist in protonated (free acid) form,
b) applying the mixture obtained in step a) on or contacting the mixture obtained in step a) with a weakly basic macroporous anion exchange resin, to provide a solution enriched in the second oligosaccharide, and
c) applying the solution enriched in the second oligosaccharide from step b) on or contacting said eluate with a basic anion exchange resin, such as a weakly basic anion exchange resin of the gel type.

By using step c) of the method, obtention of the second oligosaccharide in high purity is possible.

Accordingly, the invention also relates to a method of separating a second oligosaccharide containing at least one sialyl group from a mixture comprising a first oligosaccharide containing at least one sialyl group, said first oligosaccharide and optionally a neutral oligosaccharide, wherein said first oligosaccharide contains at least one monosaccharide unit less than the second oligosaccharide, comprising the steps of:
a) providing said mixture in a solvent with a pH level to ensure that at least 90% of the sialyl groups of the first and the second oligosaccharides exist in protonated (free acid) form,
b) applying the mixture on or contacting the mixture with a weakly basic macroporous anion exchange resin, ensuring the binding of the first oligosaccharide to the resin and thereby providing a solution enriched in the second oligosaccharide and optionally the neutral oligosaccharide,
c) applying the solution of step b) on or contacting the solution of step b) with a basic anion exchange resin, such as a weakly basic anion exchange resin of the gel type, ensuring the binding of the second oligosaccharide to the resin and thereby optionally eluting the neutral oligosaccharide,
d) eluting the second oligosaccharide from the resin, and
e) isolating the second oligosaccharide from the eluate of step d).

### Definitions

The term “oligosaccharides” preferably means carbohydrate polymers having a linear or branched structure containing a plurality of, but at least two, monosaccharide units connected together by interglycosidic linkages. In the context of the present invention, oligosaccharides also include disaccharides. The oligosaccharides in the context of the present invention are preferably in free form, i.e. they do not contain a protective group on any of their free anomeric, primary and secondary OH-groups (e.g. an ether, ester, acetal, etc.), and—in aminodeoxy sugars—they do not contain a protective group on their free NH₂-groups other than acetyl. The oligosaccharides are preferably di-, tri-, tetra-, penta- or hexasaccharides. The term “monosaccharide” preferably means a sugar (carbohydrate) of 5-9 carbon atoms that is an aldose (e.g. D-glucose, D-galactose, D-mannose, D-ribose, D-arabinose, L-arabinose, D-xylose, etc.), a ketose (e.g. D-fructose, D-sorbose, D-tagatose, etc.), a deoxysugar (e.g. L-rhamnose, L-fucose, etc.), a deoxy-aminosugar (e.g. N-acetylglucosamine, N-acetylmannosamine, N-acetylgalactosamine, etc.), an uronic acid, an aldonic acid, a ketoaldonic acid (e.g. sialic acid), an aldaric acid or a sugar alcohol.

The term “oligosaccharide containing at least one carboxylic acid group” preferably means an oligosaccharide having a monosaccharide unit containing a carboxylic acid group. The monosaccharide unit containing a carboxylic acid group is preferably a uronic acid, an aldonic acid, a ketoaldonic acid or an aldaric acid, more preferably a ketoaldonic acid. The ketoaldonic acid is preferably a neuraminic acid such as N-acetyl-, glycolyl- or deamino-neuraminic acid (KDN), more preferably N-acetyl-neuraminic acid (NANA, sialic acid, Neu5Ac). Accordingly, the NANA-containing oligosaccharides may be also referred to as “sialylated oligosaccharides”. Thus, in one embodiment, the first and second oligosaccharides containing at least one carboxylic acid group are sialylated oligosaccharides. Preferably, both the first and the second oligosaccharides contain only one carboxylic acid group, and more preferably only one sialic acid unit.

The term “human milk oligosaccharide” or “HMO”, as used herein, unless otherwise specified, refers generally to a number of complex carbohydrates found in human breast milk (see e.g. (Urashima et al.: Milk Oligosaccharides, Nova Biomedical Books, New York, 2011; Chen Adv. Carbohydr. Chem. Biochem. 72, 113 (2015)), that can be in acidic or neutral form. Acidic HMOs, referred to also as “sialylated human milk oligosaccharides” or “sialylated HMOs” or “charged HMOs”, contain at least one sialic acid unit, preferably only one sialic acid unit. Examples include 3′-sialyllactose (3′-SL), 6′-sialyllactose (6′-SL), sialyllacto-N-tetraose-a (LST-a), sialyllacto-N-tetraose-b (LST-b), sialyllacto-N-tetraose-c (LST-c), and 3-fucosyl-3′-sialyl-lactose (FSL).

Throughout the present text, the terms “the first oligosaccharide containing at least one carboxylic acid group” and “the first oligosaccharide” are used interchangeably. The same applies to the terms “the second oligosaccharide containing at least one carboxylic acid group” and “the second oligosaccharide”.

### A Mixture Comprising at Least a First Oligosaccharide and a Second Oligosaccharide, Both Containing at Least One Carboxylic Acid Group

The second oligosaccharide in the method of the present invention contains at least one additional monosaccharide compared to the first oligosaccharide, with other words, the polymerization degree of the second oligosaccharide is higher than that of the first oligosaccharide. In one embodiment, the first oligosaccharide is a disaccharide and the second oligosaccharide is a tri-, tetra-, penta-, hexa- or higher oligosaccharide. In other embodiment, the first oligosaccharide is a trisaccharide and the second oligosaccharide is a tetra-, penta-, hexa- or higher oligosaccharide. In other embodiment, the first oligosaccharide is a tetrasaccharide and the second oligosaccharide is a penta-, hexa- or higher oligosaccharide. In other embodiment, the first oligosaccharide is a pentasaccharide and the second oligosaccharide is a hexa- or higher oligosaccharide. Moreover, in more preferred embodiments, the second oligosaccharide contains only (exactly) one additional monosaccharide compared to the first oligosaccharide. In this regard, when the first oligosaccharide is a disaccharide, the second oligosaccharide is a trisaccharide; when the first oligosaccharide is a trisaccharide, the second oligosaccharide is a tetrasaccharide; when the first oligosaccharide is a tetrasaccharide, the second oligosaccharide is a pentasaccharide; or when the first oligosaccharide is a pentasaccharide, the second oligosaccharide is a hexasaccharide; and so on. In other preferred embodiments, the second oligosaccharide contains exactly two additional monosaccharides compared to the first oligosaccharide. In this regard, when the first oligosaccharide is a disaccharide, the second oligosaccharide is a tetrasaccharide; when the first oligosaccharide is a trisaccharide, the second oligosaccharide is a pentasaccharide; when the first oligosaccharide is a tetrasaccharide, the second oligosaccharide is a hexasaccharide; and so on. In other preferred embodiments, the second oligosaccharide contains exactly three additional monosaccharides compared to the first oligosaccharide. In this regard, when the first oligosaccharide is a disaccharide, the second oligosaccharide is a pentasaccharide; when the first oligosaccharide is a trisaccharide, the second oligosaccharide is a hexasaccharide; and so on. Even more preferably, in any of the above recited preferred or more preferred embodiments, the first and the second oligosaccharide contain only one carboxylic acid group, particularly only one sialic acid unit.

The method of the invention is typically useful when the second oligosaccharide is a product of an incomplete transfer of a sialic acid unit from a sialylated di-, tri- or higher saccharide donor (as first oligosaccharide) to a di-, tri-, tetra- or higher oligosaccharide acceptor by using a trans-sialidase, wherein the acceptor oligosaccharide is preferably a neutral oligosaccharide (not containing sialic acid). Accordingly, in one embodiment, the first oligosaccharide is a disaccharide or a trisaccharide. In another embodiment, the first oligosaccharide is selected from 3′-sialyllactose (3′-SL) and 6′-sialyllactose (6′-SL).

In one embodiment, a mixture comprising at least a first oligosaccharide and a second oligosaccharide, both containing at least one carboxylic acid group, may be produced by fermentation.

In a trans-sialidase mediated enzymatic reaction mentioned above, the oligosaccharide accepting the sialic acid unit is typically a di-, tri-, tetra-, penta- or higher oligosaccharide that preferably does not contain a sialic acid unit. In general, the sialylated oligosaccharide donor (that is the first oligosaccharide in the context of the present invention) does not contain more monosaccharide units than the acceptor oligosaccharide. Accordingly, the product of the reaction (that is the second oligosaccharide in the context of the present invention) is an oligosaccharide that contains exactly one monosaccharide unit more (which is a sialic acid unit) than the acceptor oligosaccharide; in this regard the second oligosaccharide comprises the structure of the oligosaccharide acceptor.

The mixture of the first and the second oligosaccharides in the context of the present invention is thus typically the result of an incomplete transfer by trans-sialidase of a sialic acid unit from the sialylated oligosaccharide donor to a neutral oligosaccharide acceptor. Hence, in one embodiment of the method according to the present invention, the mixture of the first and second oligosaccharides is prepared by adding a trans-sialidase to a mixture containing the first oligosaccharide and a precursor oligosaccharide substrate (acceptor) that does not contain a carboxylic or sialic acid group, thereby transferring the sialyl acid unit from the first oligosaccharide to the acceptor and thus making the second oligosaccharide. The trans-sialidase mediated enzymatic reaction can be depicted as follows:

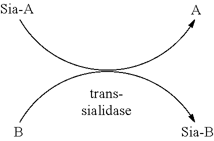

wherein Sia-A is an embodiment of the first oligosaccharide and being a di- tri- or higher sialylated oligosaccharide, Sia is the sialic acid unit or moiety, compound B is a di-, tri-, tetra-, penta- or higher oligosaccharide acceptor that preferably does not contain a sialic acid unit, Sia-B is an embodiment of the second oligosaccharide and being a tri-, tetra-, penta- or higher sialylated oligosaccharide and compound A is a leaving mono- or oligosaccharide from Sia-A, that is the desialylated Sia-A. The transsialidases, in general, are able to transfer the Sia residue from the newly formed Sia-B back to the compound A that has previously been produced from Sia-A, therefore reaching an equilibrium: B+Sia-A [symbol] Sia-B+A. In the above enzymatic reaction system, if Sia-A and compound B have the same monosaccharide units in their structure, Sia-B contains exactly one monosaccharide unit more than Sia-A (thus, if both Sia-A and compound B are trisaccharides, Sia-B is a tetrasaccharide, and so on). If Sia-A and compound B have the same monosaccharide units in their structure, Sia-B contains exactly one monosaccharide unit more than Sia-A (thus, if both Sia-A and compound B are trisaccharides, Sia-B is a tetrasaccharide, and so on). If Sia-A contains exactly one monosaccharide unit less than compound B, Sia-B contains exactly two monosaccharide units more than Sia-A (thus, if Sia-A is a trisaccharide and compound B is a tetrasaccharide, Sia-B is a pentasaccharide, and so on). If Sia-A contains exactly two monosaccharide units less than compound B, Sia-B contains exactly three monosaccharide units more than Sia-A (thus, if Sia-A is a trisaccharide and compound B is a pentasaccharide, Sia-B is a hexasaccharide, and so on).

The transsialidases show a selectivity towards the donors and to the product. Thus, an α2,3-transsialidase favourably transfers the sialic acid group from an α2,3-sialylated donor and makes preferably an α2,3-sialylated product. Accordingly, the term “α2,3-transsialidase” preferably means any wild type or engineered sialidase that is able to transfer a sialyl residue of a preferably α2,3-sialylated donor to the 3-position of a, preferably terminal, galactose unit in an oligosaccharide acceptor. Such a transsialidase is preferably the α2,3-transsialidase from Trypanosoma cruzi (TcTS). Similarly, an α2,6-transsialidase favourably transfers the sialic acid group from an α2,6-sialylated donor and makes preferably an α2,6-sialylated product. Accordingly, the term “α2,6-transsialidase” preferably means any wild type or engineered sialidase that is able to transfer a sialyl residue of a preferably α2,6-sialylated donor to the 6-position of a, preferably terminal, galactose unit in an oligosaccharide acceptor. Such transsialidases are preferably those disclosed in WO 2016/199069, the content of which is incorporated herein by reference in its entirety.

The present invention, in one embodiment, provides a convenient method to separate Sia-A from the reaction milieu comprising Sia-A, Sia-B, A and B, optionally followed by the separation of Sia-B from the neutral oligosaccharides A and B.

A mixture comprising at least a first oligosaccharide containing a sialic acid unit (Sia-A) and a second oligosaccharide containing a sialic acid unit (Sia-B) can be produced e.g. in accordance with WO 2016/157108 or WO 2016/199071, the contents of which are incorporated herein by reference in their entirety.

Preferably, for making the mixture of the first and second oligosaccharides in a trans-sialidase mediated enzymatic reaction, the precursor oligosaccharide substrate (acceptor, compound B) is a neutral HMO. Advantageously, the precursor oligosaccharide substrate (acceptor) is 3-FL, LNT, LNnT, LNFP-II or LNFP-VI.

Accordingly, in one embodiment, the present invention relates to a method of separating a first oligosaccharide containing a sialic acid unit (referred to as Sia-A) from a mixture comprising said first oligosaccharide and a second oligosaccharide containing a sialic acid unit (referred to as Sia-B), compound A and compound B, wherein said first oligosaccharide contains at least one monosaccharide unit less than the second oligosaccharide, comprising the steps of:
a) providing said mixture in a solvent with a pH level to ensure that at least 90% of the sialic acid units of Sia-A and Sia-B exist in protonated (acid) form, and
b) applying the mixture of step a) on or contacting the mixture of step a) with a weakly basic macroporous anion exchange resin, preferably to bind Sia-A and provide an aqueous solution enriched in Sia-B and containing compounds A and B.

In one embodiment, the method further comprises step c): applying the aqueous solution from step b) on or contacting said solution with a basic anion exchange resin, such as a weakly basic anion exchange resin of the gel type, preferably to bind Sia-B and provide an aqueous solution enriched in compounds A and B.

In one embodiment, Sia-A is selected from the group consisting of 3′-SL and 6′-SL.

In one embodiment, Sia-B is selected from the group consisting of FSL (3-O-fucosyl-3′-O-sialyllactose, Neu5Acα(2-3)-Galβ(1-4)-[Fucα(1-3)-]Glc), LST-a (sialyllacto-N-tetraose a, Neu5Acα(2-3)-Galβ(1-3)-GlcNAcβ(1-3)-Galβ(1-4)-Glc), LST-c (sialyllacto-N-tetraose c, Neu5Acα(2-6)-Galβ(1-4)-GlcNAcβ(1-3)-Galβ(1-4)-Glc), Neu5Acα(2-6)-Galβ(1-3)-GlcNAcβ(1-3)-Galβ(1-4)-Glc, Neu5Acα(2-3)-Galβ(1-4)-GlcNAcβ(1-3)-Galβ(1-4)-Glc, F-LST-a (Neu5Acα(2-3)-Galβ(1-3)-[Fucα(1-4)-]GlcNAcβ(1-3)-Galβ(1-4)-Glc)) and F-LST-c (Neu5Acα(2-6)-Galβ(1-4)-GlcNAcβ(1-3)-Galβ(1-4)-[Fucα(1-3)-]Glc)).

In one embodiment, Sia-A is selected from the group consisting of 3′-SL and 6′-SL, and Sia-B is selected from the group consisting of FSL (3-O-fucosyl-3′-O-sialyllactose, Neu5Acα(2-3)-Galβ(1-4)-[Fucα(1-3)-]Glc), LST-a (sialyllacto-N-tetraose a, Neu5Acα(2-3)-Galβ(1-3)-GlcNAcβ(1-3)-Galβ(1-4)-Glc), LST-c (sialyllacto-N-tetraose c, Neu5Acα(2-6)-Galβ(1-4)-GlcNAcβ(1-3)-Galβ(1-4)-Glc), Neu5Acα(2-6)-Galβ(1-3)-GlcNAcβ(1-3)-Galβ(1-4)-Glc, Neu5Acα(2-3)-Galβ(1-4)-GlcNAcβ(1-3)-Galβ(1-4)-Glc, F-LST-a (Neu5Acα(2-3)-Galβ(1-3)-[Fucα(1-4)-]GlcNAcβ(1-3)-Galβ(1-4)-Glc)) and F-LST-c (Neu5Acα(2-6)-Galβ(1-4)-GlcNAcβ(1-3)-Galβ(1-4)-[Fucα(1-3)-]Glc)).

In one embodiment, the first oligosaccharide (Sia-A) is 6′-SL and the second oligosaccharide c(Sia-B) is LST-c (sialyllacto-N-tetraose c, Neu5Acα(2-6)-Galβ(1-4)-GlcNAcβ(1-3)-Galβ(1-4)-Glc), preferably obtained from the following α2,6-transsialidase catalysed reaction: 6′-SL+LNnT [symbol] LST-c+lactose.

In one embodiment, the first oligosaccharide (Sia-A) is 3′-SL and the second oligosaccharide (Sia-B) is LST-a (sialyllacto-N-tetraose a, Neu5Acα(2-3)-Galβ(1-3)-GlcNAcβ(1-3)-Galβ(1-4)-22 Glc), preferably obtained from the following α2,3-transsialidase catalysed reaction: 3′-SL+LNT [symbol] LST-a+lactose.

In one embodiment, the first oligosaccharide (Sia-A) is 3′-SL and the second oligosaccharide (Sia-B) is FSL (3-O-fucosyl-3′-O-sialyllactose, Neu5Acα(2-3)-Galβ(1-4)-[Fucα(1-3)-]Glc), preferably obtained from the following α2,3-transsialidase catalysed reaction: 3′-SL+3-FL [symbol] FSL+lactose.

In one embodiment, the first oligosaccharide is (Sia-A) 3′-SL and the second oligosaccharide (Sia-B) is a F-LST-a (Neu5Acα(2-3)-Galβ(1-3)-[Fucα(1-4)-]GlcNAcβ(1-3)-Galβ(1-4)-Glc)), preferably obtained from the following α2,3-transsialidase catalysed reaction: 3′-SL+LNFP-∥ [symbol] F-LST-a+lactose.

In one embodiment, the first oligosaccharide is (Sia-A) 6′-SL and the second oligosaccharide (Sia-B) is a F-LST-c (Neu5Acα(2-6)-Galβ(1-4)-GlcNAcβ(1-3)-Galβ(1-4)-[Fucα(1-3)-]Glc)), preferably obtained from the following α2,6-transsialidase catalysed reaction: 6′-SL+LNFP-VI [symbol] F-LST-c+lactose.

In one embodiment, the first oligosaccharide is (Sia-A) 6′-SL and the second oligosaccharide (Sia-B) is Neu5Acα(2-6)-Galβ(1-3)-GlcNAcβ(1-3)-Galβ(1-4)-Glc, preferably obtained from the following α2,6-transsialidase catalysed reaction: 6′-SL+LNT [symbol] Neu5Acα(2-6)-Galβ(1-3)-GlcNAcβ(1-3)-Galβ(1-4)-Glc+lactose.

In one embodiment, the first oligosaccharide is (Sia-A) 3′-SL and the second oligosaccharide (Sia-B) is Neu5Acα(2-3)-Galβ(1-4)-GlcNAcβ(1-3)-Galβ(1-4)-Glc, preferably obtained from the following α2,3-transsialidase catalysed reaction: 3′-SL+LNnT [symbol] Neu5Acα(2-3)-Galβ(1-4)-GlcNAcβ(1-3)-Galβ(1-4)-Glc+lactose.

Providing the Mixture with the Correct pH (Step a)

In step a) of the method according to the present invention, the mixture is provided with pH at a level adapted to the specific oligosaccharides to be separated in the method. In this regard, the mixture is preferably an aqueous solution. In order for the separation to be optimal, the carboxylic acid groups of the first and second oligosaccharide should predominantly be in protonated form, i.e. at least 90% of the carboxylic acid groups should be in free acid form. The skilled person knows how to adjust pH in order to ensure the required level of the protonated, free acid form. As an example, the pKₐ of the carboxylic acid containing oligosaccharide may be determined and the required pH would then be calculated using the Henderson-Hasselbalch equation. In order to have the required amount of the protonated form (90%), the pH would be calculated as pH≈pKₐ−0.954.

In one embodiment, at least 92% of the carboxylic acid groups are in protonated form. In another embodiment, at least 95% of the carboxylic acid groups are in protonated form. In still another embodiment, at least 98% of the carboxylic acid groups are in protonated form.

The pH may in principle be adjusted by any method known to the skilled person, such as e.g. using a stronger acid than the carboxylic acid group containing first and second oligosaccharides, preferably a stronger inorganic acid, the exemplary embodiments of which may be a HCl-solution or a sulfuric acid solution. In one embodiment, the pH is set to around 1.5-3.

A convenient and also a preferred way of achieving the pH adjustment in view of step a) of the method according to the present invention is to use a protonated cation exchange resin. Accordingly, in one embodiment, the pH-set mixture provided in step a) is provided by applying the mixture of the first and the second oligosaccharide on or contacting said mixture with a protonated acidic cation exchange resin (an acidic cation exchange resin in H⁺-form). Preferably, the protonated acidic cation exchange resin is a protonated strong acidic cation exchange resin.

I one embodiment, the pH-set mixture in the form of an aqueous solution provided in step a) can be obtained by loading an aqueous solution containing the first and the second oligosaccharide on the top of a column filled with a protonated acidic cation exchange resin, preferably a strong acidic cation exchange resin, eluting with water and collecting the fractions containing the first and second acidic oligosaccharides in protonated form (eluate). The amount of the acidic cation exchange resin shall be sufficient to convert the first and the second acidic oligosaccharide to protonated form e.g. from their corresponding salt forms. In an alternative embodiment, an aqueous solution containing the first and the second oligosaccharide is contacted with a protonated acidic cation exchange resin, preferably a strong acidic cation exchange resin, in a vessel under or without agitation until substantially all carboxylic acid groups are converted into protonated form. The resin is then separated e.g. by filtration (filtrate). Both the filtrate and the eluate obtainable in step a) may be referred to as a “pH-set mixture”, a “pH-set (aqueous) solution”, an “acidic cation exchange resin treated mixture” or an “acidic cation exchange resin treated (aqueous) solution”. Said pH-set solution is ready to be used for step b) of the invention.

In any of embodiments above, the mixture comprising at least a first oligosaccharide and a second oligosaccharide, both containing at least one carboxylic acid group, preferably a sialic acid unit or moiety, may further comprise neutral oligosaccharides. The neutral oligosaccharides do not bind to the acidic cation exchange resin, therefore are to be collected together with the acidified (protonated) first and second oligosaccharides after step a).

Furthermore, the mixture comprising at least a first oligosaccharide and a second oligosaccharide, both containing at least one carboxylic acid group, preferably a sialic acid unit or moiety, and optionally a neutral oligosaccharide, may further comprise inorganic anions of a strong inorganic acid, typically chloride, sulphate, nitrate, phosphate and the like. Their presence is tolerable as long as they do not substantially reduce the capacity of the of weakly basic macroporous anion exchange resin with regard to the first oligosaccharide containing at least as carboxylic group used in step b) (vide infra). Suitably, if the mixture of the first and the second oligosaccharide, both containing at least one carboxylic acid group, obtained from an enzymatic reaction such as those disclose above, the amount of inorganic anions does not substantially influence the separation of the first oligosaccharide from the second oligosaccharide in step b) of the present invention. In any event, if the presence of such inorganic anions is not desirable, they may be at least partially removed from the mixture comprising the first oligosaccharide and the second oligosaccharide, both containing at least one carboxylic acid group, preferably a sialic acid unit or moiety, before applying the steps of the invention on the mixture, for example by utilization of suitable membranes that retain the first and the second oligosaccharide and allow the inorganic anions to pass because of their substantially smaller size compared to the first and the second oligosaccharide.

In the embodiment wherein an acid, preferably an inorganic acid, stronger than any of the first and the second oligosaccharide, both containing at least one carboxylic acid group, preferably a sialic acid unit or moiety, is used to set the pH of the solution containing the first and the second oligosaccharide to the desired value, that is when at least 90% of the carboxylic acid groups in the first and the second oligosaccharide are in protonated form, it is desirable if the acid is not applied in too much excess in order that the amounts of the acid do not substantially reduce the capacity of the of weakly basic macroporous anion exchange resin with regard to the first oligosaccharide containing at least as carboxylic group used in step b) (vide infra). To avoid such an excess use of an inorganic acid, it is advisable that the pH is set to around 1.5-3.

### Applying the Mixture Obtained in Step a) on a Weakly Basic Macroporous Anion Exchange Resin (Step b)

In step b) of the method according to the present invention, the pH-set mixture in the form of an aqueous solution provided in step a) is applied to or contacted with a weakly basic macroporous anion exchange resin.

Basic anion exchange resins may be strongly or weakly basic and may be macroporous or of the gel type. Macroporous ion exchange resins are designed with a degree of crosslinking allowing larger pores in the three-dimensional structure, whereas ion exchange resins of the gel type do not contain the larger pores.

The basic anion exchange resins typically have a polyacrylic or polystyrene backbone, which are crosslinked between the individual polymer chains. A typical crosslinker is divinylbenzene (DVB). Accordingly, in one preferred embodiment, the weakly basic macroporous anion exchange resin comprises a polystyrene backbone. In another embodiment, the weakly basic macroporous anion exchange resin comprises a backbone crosslinked by divinylbenzene. In still another preferred embodiment, the weakly basic macroporous anion exchange resin comprises a divinylbenzene-crosslinked polystyrene backbone.

Weakly basic anion exchange resins typically contain base groups having a lone pair of electrons to attract proton, such as certain nitrogen containing groups. The base groups shall not be in protonated form, in other words, they are free bases. Hence, in one embodiment, the weakly basic macroporous anion exchange resin contains base groups having a lone pair of electrons to attract proton. In a further embodiment, the weakly basic macroporous anion exchange resin contains a nitrogen atom having a lone pair of electrons to attract proton. Such groups include e.g. a primary amines, a secondary amine, a tertiary amine (free amine groups), a guanidino or a nitrogen containing heteroaromatic group (like pyridino, pyrimidino, etc.), preferably a tertiary amine. In still a further embodiment, the weakly basic macroporous anion exchange resin contains free amine groups on a divinylbenzene-crosslinked polystyrene backbone. Examples of the latter include Lewatit MP62 from Lanxess, Dowex 77 from Dow, DIAION WA30 from Mitsubishi Chemical, and Dowex 66 from Dow.

Without being bound by a particular theory, the basicity and pore size of the weakly basic macroporous anion exchange resins in free base form allow a selective binding of the first oligosaccharide containing at least one carboxylic acid group relative to the second oligosaccharide containing at least one carboxylic acid group. As with any other resin, weakly basic macroporous anion exchange resins have a certain binding capacity. Accordingly, the loaded amount of oligosaccharides on the resin is advantageously adjusted according to the binding capacity/saturation limit towards the best binding oligosaccharide, i.e. the first oligosaccharide in the method according to the invention. Alternatively, the amount of the weakly basic macroporous anion exchange resin is advantageously adjusted to match the loaded amount of oligosaccharides to the resin according to the binding capacity/saturation limit of the resin towards the best binding oligosaccharide, i.e. the first oligosaccharide. Thus, in one embodiment of the method according to the present invention, the amount of the first oligosaccharide is around a previously determined saturation limit for the first oligosaccharide concerning the weakly basic macroporous anion exchange resin. The saturation limit can be determined by passing a sample with a relatively high amount of the first oligosaccharide through the resin and measure how much passes through the resin. The saturation limit is calculated as the initial amount minus the amount that passes through the resin.

In one embodiment, the amount of the first oligosaccharide in the mixture is around 80-120% of the previously determined saturation limit for the first oligosaccharide concerning the weakly basic macroporous anion exchange resin, such as 85%, 90%, 95%, 100%, 105%, 110% or 115%.

The presence of acids stronger than the first oligosaccharide, typically inorganic acids, in the feed solution may occupy the free base functional groups of the weakly basic macroporous anion exchange resins used in step b). However, their presence do not substantially influence the separation effect of step b) of the invention if their amounts are minor, e.g. if the mixture of the first and the second oligosaccharide was obtained from an enzymatic reaction (see above) and step a) is conducted with using a strong acidic ion exchange resin (in H⁺-form) or the strong acid used in step a) to covert the first and the second oligosaccharides comprising a carboxylic acid group to protonated form is not applied in excess.

I one embodiment, the pH-set mixture in the form of an aqueous solution obtained in step a) can be loaded on the top of a column filled with a calculated amount of the weakly basic macroporous anion exchange resin, preferably the weakly basic macroporous anion exchange resin having a divinylbenzene-crosslinked polystyrene backbone and eluting with water. The first oligosaccharide binds to the weakly basic macroporous anion exchange resin by adsorption to the free basic functional groups of the resin and the second oligosaccharide (together with other neutral oligosaccharides that are optionally present) goes through the resin and is collected as eluate.

In an alternative embodiment, the pH-set mixture in the form of an aqueous solution obtained in step a) is contacted with a calculated amount of the weakly basic macroporous anion exchange resin, preferably the weakly basic macroporous anion exchange resin having a divinylbenzene-crosslinked polystyrene backbone, in a vessel under or without agitation until substantially all first oligosaccharide binds to the weakly basic macroporous anion exchange resin by adsorption to the free basic functional groups of the resin. The second oligosaccharide (together with other neutral oligosaccharides that are optionally present) remains in solution. The resin with the first oligosaccharide bound to it is then separated, e.g. by filtration, from the solution containing the second oligosaccharide (filtrate). Both the filtrate and eluate obtainable in step b) may be referred to as an (aqueous) solution enriched in the second oligosaccharide.

Optionally, after conducting step b) of the present invention and collecting the solution enriched in the second oligosaccharide, the first oligosaccharide can then be eluted from the weakly basic macroporous anion exchange resin with an appropriate second eluting solution, e.g. with diluted ammonia solution or a solution of an acid that is a stronger acid than the first oligosaccharide, preferably an inorganic acid such as HCl, in a continuous or batch mode. Accordingly, the first oligosaccharide can then be separated from the second oligosaccharide in a sufficient purity and may be isolated in a syrupy form or by e.g. crystallization, precipitation, spray-drying, freeze-drying.

The skilled person understands that, depending on the conditions, some minor amounts of the first oligosaccharide may not bind to the weakly basic macroporous anion exchange resin and/or some minor amounts of the second oligosaccharide may bind to the weakly basic macroporous anion exchange resin. Accordingly, if not a complete separation of the first oligosaccharide from the second oligosaccharide is achievable, but at least the majority of the first oligosaccharide can be separated from at least the majority of the second oligosaccharide.

In this regard, a fraction enriched in the second oligosaccharide can be collected at the end of step b), and subsequently at least an enriched fraction of the first oligosaccharide can be washed off from the weakly basic macroporous anion exchange resin with the second eluting solution.

### Optional Step c)

The method according to the present invention serves to separate the first and second oligosaccharides. While the first oligosaccharide is typically available from other sources in high purity, the present method allows for isolation of the second oligosaccharide in degrees of purity that would otherwise require low-throughput chromatographic methods, such as gel chromatography or preparative HPLC. Hence, in one embodiment of the method of the present invention, the solution containing and enriched in the second oligosaccharide resulting from step b) is collected from which the second oligosaccharide may be isolated.

In one embodiment, the second oligosaccharide may directly be isolated from the aqueous solution obtained in step b) in syrupy form or by the methods known in the art, including crystallization, precipitation, spray-drying, freeze-drying etc.

In other embodiment, the second oligosaccharide may be further purified and then isolated from the aqueous solution obtained in step b). Accordingly, said solution is applied on or contacted with a basic anion exchange resin, preferably a weak basic anion exchange resin in base form, ensuring the oligosaccharide to bind to the resin. The weak basic ion exchange resin applied in the optional step c) may or may not be identical with the weak basic ion exchange resin applied in the precedent step b). Preferably, the weak basic ion exchange resin applied in step c) is not identical with the weak basic ion exchange resin applied in step b) More preferably, the weak basic anion exchange resin applied in step c) is of the gel type. Even more preferably, the weak basic anion exchange resin of the gel type is a polyacrylic resin.

The initial mixture of the method according to the present invention comprises at least the first and second oligosaccharides containing at least one carboxylic acid group. The mixture may in addition also contain further oligosaccharides that do not contain any carboxylic acid group (“neutral oligosaccharides”). In step c) of the method according to the present invention, if the solution obtained in step b) in addition to the second oligosaccharide also comprises neutral oligosaccharides, the neutral oligosaccharides do not bind to the resin whereas the second oligosaccharide does, thereby the neutral oligosaccharides are conveniently separated from the second oligosaccharide. After collecting the eluate or the filtrate (depending on the way whether step c) is performed in chromatography or batch mode) containing the neutral oligosaccharides, the bound second oligosaccharide can then be eluted from the basic resin with an appropriate eluent, e.g. with diluted ammonia solution or a solution of an acid that is a stronger acid than the second oligosaccharide, preferably an inorganic acid such as HCl.

If step a) of the method according to the present invention is carried out using a protonated cation exchange resin, steps a) and b) may conveniently be carried out without any intermediate collection of eluate fractions by directly passing the eluate from step a) to the resin in step b). Similarly, if step c) is necessary, the eluate from step b) may conveniently be passed directly to the resin in step c). Hence, in one embodiment of the method according to the present invention, the steps are carried out without any intermediate collection of eluate fractions.

The following numbered aspects of the invention are provided:

Aspect 1. A method of separating a first oligosaccharide containing at least one carboxylic acid group from a mixture comprising at least said first oligosaccharide and a second oligosaccharide containing at least one carboxylic acid group, wherein said first oligosaccharide contains at least one monosaccharide unit less than the second oligosaccharide, comprising the steps of:
a) providing said mixture in a solvent, preferably water, with a pH level to ensure that at least 90% of the carboxylic acid groups of the first and the second oligosaccharides exist in protonated (acid) form, and
b) contacting the mixture of step a) with a weakly basic macroporous anion exchange resin in free base form, thereby providing an aqueous solution enriched in the second oligosaccharide.

Aspect 2. The method of aspect 1, wherein the oligosaccharides are sialylated human milk oligosaccharides, preferably monosialylated human milk oligosaccharides.

Aspect 3. The method of aspect 1 or 2, wherein the macroporous resin comprises polystyrene backbone structure, preferably crosslinked with divinyl-benzene.

Aspect 4. The method of any of aspects 1 to 3, wherein the mixture of step a) contacted with the weakly basic macroporous anion exchange resin in step b) contains the first oligosaccharide in an amount that is around a previously determined saturation limit for the first oligosaccharide concerning the weakly basic macroporous anion exchange resin, preferably 80-120% of the previously determined saturation limit.

Aspect 5. The method of any of aspects 2 to 4, wherein the mixture of the first and second oligosaccharides is prepared by adding a trans-sialidase to the first oligosaccharide and a precursor oligosaccharide substrate that does not contain a carboxylic acid group.

Aspect 6. The method of aspect 5, wherein, in step c), the aqueous solution obtained in step b) that is enriched in the second oligosaccharide and contains the precursor oligosaccharide is contacted with an anion exchange resin, preferably a weakly basic anion exchange resin in free base form.

Aspect 7. The method of aspect 6, wherein the weakly basic anion exchange resin is of a gel type.

Aspect 8. The method of any of the preceding aspects, wherein the pH in step a) is 1.5-3.

Aspect 9. The method of any of the preceding aspects, wherein the first oligosaccharide is 3′-sialyllactose and the second oligosaccharide is FSL (3-O-fucosyl-3′-O-sialyllactose), LST-a (sialyllacto-N-tetraose a), F-LST-a (Neu5Acα(2-3)-Galβ(1-3)-[Fucα(1-4)-]GlcNAcβ(1-3)-Galβ(1-4)-Glc)) or Neu5Acα(2-3)-Galβ(1-4)-GlcNAcβ(1-3)-Galβ(1-4)-Glc.

Aspect 10. The method of any of the aspects 1 to 8, wherein the first oligosaccharide is 6′-sialyllactose and the second oligosaccharide is LST-c (sialyllacto-N-tetraose c), F-LST-c (Neu5Acα(2-6)-Galβ(1-4)-GlcNAcβ(1-3)-Galβ(1-4)-[Fucα(1-3)-]Glc)) or Neu5Acα(2-6)-Galβ(1-3)-GlcNAcβ(1-3)-Galβ(1-4)-Glc.

### EXAMPLES

### Example 1—Enzymatic Reaction and Purification of LST-c

Essentially in accordance with Example 1 of WO 2016/199071, LNnT (136 mmol) and 6′-SL (72 mmol) were reacted in the presence of the A218Y-N222R-G349S-S412P-D451K mutant of Photobacterium leiognathi JT-SHIZ-119 sialyl transferase truncated by its signal peptide (Δ2-15) disclosed therein. After purification and freeze-drying, the following mixture was obtained: LNnT (45.2 w %), 6′-SL (16.3 w %), LST-c (31.4 w %) and lactose (9.6 w %). The mixture was dissolved in water to give a 3.6 Brix (° Bx) solution and passed through three interconnected columns filled with different ion exchange resins so that the eluate from resin 1 was directly led to the top of the column of resin 2, and its eluate was directly led to the top of the column of resin 3. Resin 1 was Dowex88 (a strongly acidic cation exchange resin (SAC) in H⁺-form), while resin 2 and 3 were weakly basic anion exchange resins (WBA1: Dowex66 which is a macroporous polystyrene-DVB resin, and WBA2: Amberlite FPA53 which is a polyacrylic gel-type resin; both are in free base form). LNnT and lactose were not binding to any of the resins. The WBA1 resin was used so that it corresponded to 5 mmol 6′-SL/100 ml resin. 6′-SL was binding selectively to WBA1 while LST-c was binding selectively to WBA2. Subsequently, the columns were disconnected. LST-c was eluted from WBA2 using 0.5 M HCl-solution and the pH was adjusted to 4.8 with NaOH-solution. The solution was desalinated by nanofiltration. LST-c was isolated by freeze-drying (24.5 g) with a purity of 91.4% (LNnT 0.3 w %, 6′-SL 2.9 w %, no lactose).

### Example 2—Enzymatic Reaction and Purification of LST-a

Essentially in accordance with WO 2016/157108, LNT (134 mmol) and 3′-SL (70 mmol) in were reacted in the presence of α2,3-transsialidase from T. cruzi (TcTS). After purification, the following mixture was obtained in a form of a brix 4.0 solution: LNT (42.1 w %), 3′-SL (10.3 w %), LST-a (24.2 w %) and lactose (10.3 w %). The solution was passed through three interconnected columns filled with different ion exchange resins so that the eluate from resin 1 was directly led to the top of the column of resin 2, and its eluate was directly led to the top of the column of resin 3. Resin 1 was Dowex88 (a strongly acidic cation exchange resin (SAC) in H⁺-form), while resin 2 and 3 were weakly basic anion exchange resins (WBA1: Dowex66 which is a macroporous polystyrene-DVB resin, and WBA2: Amberlite FPA53 which is a polyacrylic gel-type resin; both are in free base form). LNT and lactose were not binding to any of the resins. The WBA resin was used so that it corresponded to 5 mmol 3′-SL/100 ml resin. 3′-SL was binding selectively to WBA1 while LST-a was binding selectively to WBA2. Subsequently, the columns were disconnected. LST-a was eluted from WBA2 using 0.5 M HCl-solution and the pH was adjusted to around 6 with NaOH-solution. The solution was desalinated by nanofiltration. LST-a was isolated by freeze-drying (38.4 g) with a purity of 91.4% (LNT 0.5 w %, 3′-SL 0.6 w %, no lactose).

### Example 3—Enrichment of LST-c from a Mixture of LST-c, 6′-SL, LNnT and Lactose Using a Macroporous Polystyrene-DVB Weakly Basic Anion (Free Amine) Resin

10 g of a freeze-dried mixture containing LST-c 13.09 g/l, 6′-SL 5.92 g/l, LNnT 17.74 g/I and lactose 4.64 g/I was dissolved with water (240 ml) to obtain a solution of <5° Bx. The feed composition was sampled and analysed by HPLC to determine the amounts of each component.

Strongly acidic ion exchange resin DOWEX88H (40 ml) and weakly basic macroporous anion exchange resin (free base) LEWATIT MP62 (40 ml) were coupled in series and the feed solution was loaded. Fractions of 50 ml were collected, in total 16 fractions were collected using pure water as eluent.

The fractions were spotted by TLC and evaluated using AcCN:NH₃:water (6:3:1) as eluent. Fractions 2-5 contained no LST-c and were pooled separately. Fractions 6-8 indicated to contain a mixture of LST-c, LNnT and lactose and were pooled separately. Fractions 9-13 indicated to contain pure LST-c and were pooled separately.

The pH was checked in the pooled fractions and typically adjusted with 1 M NaOH-solution to 4-5.5. HPLC analysis results of the pooled fractions are summarized in below:

g/l

| | LST-c | lactose | LNnT | 6′-SL |
|---|---|---|---|---|
| Feed | 13.09 | 4.64 | 17.74 | 5.92 |
| Fractions | | | | |
| 2-5 | n.a. | 4.35 | 16.81 | n.a. |
| 6-8 | 3.58 | 1.76 | 6.69 | n.a. |
| 9-13 | 2.04 | n.a. | n.a. | n.a. |

### Example 4—Enrichment of LST-c from a Mixture of LST-c, 6′-SL, LNnT and Lactose Using a Macroporous Polystyrene-DVB Weakly Basic Anion (Free Amine) Resin

Example 3 was repeated with 12 g of freeze-dried mixture in 240 ml of water using Dowex 88H (50 ml) and the weakly basic macroporous anion exchange resin (free base) Dowex 77 (50 ml). TLC was carried out using the same eluent with Fractions 1-4 indicated only minor amount of LST-c and being pooled separately. Fractions 5-16 indicated to contain a mixture of LST-c, LNnT and lactose and were pooled separately.

The pH was checked in the pooled fractions and adjusted with 1 M NaOH to 4-5.5. HPLC analysis results of the pooled fractions are summarized in below:

g/l

| | LST-c | lactose | LNnT | 6′-SL |
|---|---|---|---|---|
| Feed | 14.87 | 5.59 | 20.05 | 6.73 |
| Fractions | | | | |
| 1-4 | 0.34 | 3.23 | 12.83 | n.a. |
| 5-16 | 5.50 | 1.32 | 4.64 | 0.16 |

### Example 5—Enrichment of LST-c from a Mixture of LST-c, 6′-SL, LNnT and Lactose Using a Macroporous Polystyrene-DVB Weakly Basic Anion (Free Amine) Resin

Example 3 was repeated with 12 g of freeze-dried mixture in 240 ml of water using Dowex 88H (50 ml) and the weakly basic macroporous anion exchange resin (free base) DIAION WA 30 (50 ml). TLC was carried out using the same eluent with Fractions 2-6 indicated no presence of LST-c and being pooled separately. Fractions 7-14 indicated to contain a mixture of LST-c, LNnT and lactose and were pooled separately.

The pH was checked in the pooled fractions and adjusted with 1 M NaOH towards 4-5.5. HPLC analysis results of the pooled fractions are summarized in below:

g/l

| | LST-c | lactose | LNnT | 6′-SL |
|---|---|---|---|---|
| Feed | 14.42 | 5.35 | 20.10 | 6.69 |
| Fractions | | | | |
| 2-6 | n.a. | 4.38 | 17.33 | n.a. |
| 7-14 | 2.03 | 0.88 | 2.75 | 0.07 |

### Example 6—Determination of the Binding Capacity of a Weakly Basic Macroporous Anionic Resin

The strongly acidic ion exchange resin Dowex 88H (200 ml) and the weakly basic macroporous anion exchange resin (free base) Dowex 66 (200 ml) were coupled in series, and a feed solution of 13.0 g of 3′-SL and 13.0 g of 6′-SL dissolved in 1 l of water was loaded on the acidic ion exchange column. In total, 14 fractions eluted from the second column (Dowex 66) were collected and checked by TLC.

No 3′-SL and 6′-SL was detected in the first fractions since they were binding to Dowex 66. When the binding capacity of Dowex 66 was reached, 3′-SL and 6′-SL started to elute (from fraction 6). Fractions 6-14 were collected containing the eluted 3′-SL and 6′-SL. The fractions were analysed by ion chromatography (IC).

According to the analysis, fractions 6-14 contained 8 g of sialyllactoses, thus 18 g of sialyllactoses were adsorbed by Dowex 66 (≈15 mmol per 100 ml of Dowex 66). Based on the composition of fractions 6-14, 3′-SL binds slightly stronger to Dowex 66 than 6′-SL.

### Example 7—Separation of 3′-SL and LST-a on a Weakly Basic Macroporous Anionic Resin

Two columns filled with the strongly acidic ion exchange resin Dowex 88H (25 ml) and the weakly basic macroporous anion exchange resin (free base) Dowex 66 (25 ml), respectively, were coupled in series.

A load solution was prepared from a freeze dried powder containing 23.6 w/w % 3′-SL and 73.9 w/w % LST-a wherein the amount of 3′-SL corresponded to the binding capacity of Dowex 66 based on Example 6 (that is ≈15 mmol/100 ml). In this regard, the feed solution should have contained ≈3.7 mmol of 3′-SL. Accordingly, 10.0 g of the above freeze-dried powder (containing thus 3.7 mmol of 3′-SL and 7.4 mmol of LST-a) was dissolved in 190 ml of water. The solution was loaded on the acidic ion exchange column and the fractions eluted from the second column (Dowex 66) were collected (45-50 ml). The flow rate was 2 bed volumes per hour.

The fractions were analysed by IC. The contents of the fractions are summarized in table below:

| | | fractions | | | | | |
|---|---|---|---|---|---|---|---|
| | feed | 1 | 2 | 3 | 4 | 5 | 6 |
| LST-a [mmol] | 7.40 | — | 0.74 | 1.60 | 1.94 | 1.73 | 0.31 |
| 3′-SL [mmol] | 3.73 | — | 0.01 | 0.04 | 0.11 | 0.17 | 0.08 |
| cumulative LST-a recovery [%] | 100 | — | 10 | 32 | 58 | 81 | 85 |
| cumulative 3′-SL recovery [%] | 100 | — | 0 | 2 | 7 | 14 | 18 |
| LST-a/3′-SL molar ratio | 2:1 | — | 74:1 | 40:1 | 18:1 | 10:1 | 4:1 |

In the combined fractions 2-6, the LST-a/3′-SL ratio was 10:1. Therefore, a chromatography of a LST-a/3′-SL mixture on weakly basic macroporous anionic resin improved the LST-a/3′-SL molar ratio from 2:1 to 10:1.

### Example 8—Separation of 6′-SL and LST-c on a Weakly Basic Macroporous Anionic Resin

Two columns filled with the strongly acidic ion exchange resin Dowex 88H (25 ml) and the weakly basic macroporous anion exchange resin (free base) Dowex 66 (25 ml), respectively, were coupled in series.

A load solution was prepared by dissolving 15.0 g of a freeze dried powder containing 22.0 w/w % 6′-SL (5.2 mmol) and 33.3 w/w % LST-c (5.0 mmol) in 285 g of water. The solution was loaded on the acidic ion exchange column and the fractions eluted from the second column (Dowex 66) were collected (45-50 ml). The flow rate was 2 bed volumes per hour.

The fractions were analysed by IC. The contents of the fractions are summarized in table below:

| | | fractions | | | | | | | |
|---|---|---|---|---|---|---|---|---|---|
| | feed | 1 | 2 | 3 | 4 | 5 | 6 | 7 | 8 |
| LST-c [mmol] | 5.01 | — | 0.2 | 1.0 | 1.1 | 1.0 | 1.0 | 0.8 | 0.2 |
| 6′-SL [mmol] | 5.21 | — | 0 | 0 | 0.3 | 0.5 | 0.7 | 0.6 | 0.5 |
| cumulative LST-c recovery [%] | 100 | — | 3 | 23 | 46 | 66 | 86 | 102 | 105 |
| cumulative 6′-SL recovery [%] | 100 | — | 0 | 1 | 6 | 16 | 28 | 41 | 50 |
| LST-c/6′-SL molar ratio | 1:1 | — | 99:1 | 98:1 | 4:1 | 2:1 | 1.4:1 | | |

In this experiment, the amount of 6′-SL (20.8 mmol/100 ml) exceeded the binding capacity of Dowex 66 (15 mmol/100 ml) by around 40%. Therefore, after the saturation of Dowex 66, the not-bound 6′-SL started to elute. Nevertheless, the fractions 2-6 contained LST-c in enriched ratios.

### Example 9—Separation of 3′-SL and FSL on a Weakly Basic Macroporous Anionic Resin

Two columns filled with the strongly acidic ion exchange resin Dowex 88H (25 ml) and the weakly basic macroporous anion exchange resin (free base) Dowex 66 (25 ml), respectively, were coupled in series.

A load solution was prepared from a freeze dried powder containing 15.9 w/w % 3′-SL and 27.5 w/w % FSL wherein the amount of 3′-SL corresponded to the binding capacity of Dowex 66 based on Example 6 (that is ≈15 mmol/100 ml). In this regard, the feed solution should have contained ≈3.7 mmol of 3′-SL. Accordingly, 15.0 g of the above freeze-dried powder (containing thus 3.8 mmol of 3′-SL and 5.3 mmol of FSL) was dissolved in 285 g of water. The solution was loaded on the acidic ion exchange column and the fractions eluted from the second column (Dowex 66) were collected (45-50 ml). The flow rate was 2 bed volumes per hour.

The fractions were analysed by IC. The contents of the fractions are summarized in table below:

| | | fractions | | | | | | |
|---|---|---|---|---|---|---|---|---|
| | feed | 1 | 2 | 3 | 4 | 5 | 6 | 7 |
| FSL [mmol] | 7.40 | — | 0.01 | 0.27 | 0.77 | 0.85 | 0.87 | 0.81 |
| 3′-SL [mmol] | 3.73 | — | 0 | 0.02 | 0.05 | 0.10 | 0.20 | 0.27 |
| cumulative FSL recovery [%] | 100 | — | 0.2 | 5 | 20 | 36 | 53 | 68 |
| cumulative 3′-SL recovery [%] | 100 | — | 0 | 0.4 | 2 | 4 | 10 | 17 |
| FSL/3′-SL molar ratio | 2:1 | — | 99:1 | 14:1 | 15:1 | 8:1 | 4:1 | 3:1 |

In the combined fractions 2-7, the FSL/3′-SL ratio was 5.6:1. Therefore, a chromatography of an FSL/3′-SL mixture on a weakly basic macroporous anionic resin improved the FSL/3′-SL molar ratio from 2:1 to 5.6:1.

1. A method of separating a first oligosaccharide containing at least one carboxylic acid group from a mixture comprising at least said first oligosaccharide and a second oligosaccharide containing at least one carboxylic acid group, wherein said first oligosaccharide contains at least one monosaccharide unit less than the second oligosaccharide, comprising the steps of:
a) providing said mixture in a solvent with a pH level to ensure that at least 90% of the carboxylic acid groups of the first and the second oligosaccharides exist in protonated (acid) form, and
b) applying the mixture of step a) on or contacting the mixture of step a) with a weakly basic macroporous anion exchange resin, thereby providing an aqueous solution enriched in the second oligosaccharide.

2. The method according to claim 1, wherein the carboxylic acid group is comprised in a neuraminic acid unit or moiety.

3. The method according to claim 2, wherein the neuraminic acid unit or moiety is a sialic acid unit or moiety:

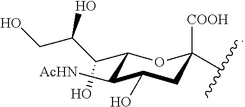

4. The method according to claim 1, wherein the first and the second oligosaccharides contain only one (single) sialic acid moiety or unit in their structures.

5. The method according to claim 1, wherein the first and the second oligosaccharides are human milk oligosaccharides (HMOs).

6. (canceled)

7. The method according to claim 1, wherein:
the first oligosaccharide is a disaccharide and the second oligosaccharide is a trisaccharide,
the first oligosaccharide is a trisaccharide and the second oligosaccharide is a tetrasaccharide,
the first oligosaccharide is a tetrasaccharide and the second oligosaccharide is a pentasaccharide, or
the first oligosaccharide is a pentasaccharide and the second oligosaccharide is a tetrasaccharide.

8. (canceled)

9. The method according to claim 7, wherein the first oligosaccharide is 3′-SL and the second oligosaccharide is FSL.

10. (canceled)

11. The method according to claim 1, wherein:
the first oligosaccharide is a disaccharide and the second oligosaccharide is a tetrasaccharide,
the first oligosaccharide is a trisaccharide and the second oligosaccharide is a pentasaccharide,
the first oligosaccharide is a tetrasaccharide and the second oligosaccharide is a hexasaccharide, or
the first oligosaccharide is a pentasaccharide and the second oligosaccharide is a pentasaccharide.

12. (canceled)

13. The method according to claim 11, wherein:
the first oligosaccharide is 3′-SL and the second oligosaccharide is LST-a,
the first oligosaccharide is 6′-SL and the second oligosaccharide is LST-c,
the first oligosaccharide is 3′-SL and the second oligosaccharide is Neu5Acα(2-3)-Galβ(1-4)-GlcNAcβ(1-3)-Galβ(1-4)-Glc, or
the first oligosaccharide is 6′-SL and the second oligosaccharide is Neu5Acα(2-6)-Galβ(1-3)-GlcNAcβ(1-3)-Galβ(1-4)-Glc.

14.-17. (canceled)

18. The method according to claim 1, wherein the second oligosaccharide is a product of a sialic acid transfer from the first oligosaccharide to an oligosaccharide acceptor mediated by a trans-sialidase.

19. (canceled)

20. The method according to claim 18, wherein the oligosaccharide acceptor is a human milk oligosaccharide (HMO).

21. The method according to claim 20, wherein the oligosaccharide acceptor is selected from 3-FL, LNT, LNnT, LNFP-II or LNFP-VI.

22. The method according to claim 1, wherein the mixture of the first and the second oligosaccharide provided in step a) is an aqueous solution; and wherein the pH of the aqueous solution is set by adding an inorganic acid stronger than any of the first and the second oligosaccharide.

23. (canceled)

24. The method according to claim 22, wherein the inorganic acid is HCl or sulfuric acid; and wherein the pH of the aqueous solution is 1.5-3.

25. (canceled)

26. The method according to claim 22, wherein the aqueous solution is provided by applying the mixture of the first and the second oligosaccharide on or contacting said mixture with a protonated acidic cation exchange resin; and wherein the acidic cation exchange resin is a strong acidic cation exchange resin.

27. (canceled)

28. The method according to claim 22, wherein, in step b), the aqueous solution provided in step a) is applied to or contacted with a weakly basic macroporous anion exchange resin.

29. The method according to claim 28, wherein the weakly basic macroporous anion exchange resin is in free base form; and wherein the aqueous solution is applied to or contacted with a weakly basic macroporous anion exchange resin to ensure that the first oligosaccharide binds to the resin thereby providing the an aqueous solution enriched in the second oligosaccharide.

30.-32. (canceled)

33. The method according to claim 28, wherein the aqueous solution applied to or contacted with the weakly basic macroporous anion exchange resin in step b) contains the first oligosaccharide in an amount that is around a previously determined saturation limit for the first oligosaccharide concerning the weakly basic macroporous anion exchange resin; and wherein the amount of the first oligosaccharide in the solution is around 80-120% of the previously determined saturation limit.

34. (canceled)

35. The method according to claim 28, wherein, in step c), the aqueous solution enriched in the second oligosaccharide and obtained in step b) is applied to or contacted with a basic anion exchange resin; and wherein the basic anion exchange resin is in base form.

36. (canceled)

37. The method according to claim 35, wherein the aqueous solution is applied to or contacted with the basic anion exchange resin to ensure that the second oligosaccharide binds to the resin.

38. The method according to claim 37, wherein the anion exchange resin is a weak basic anion exchange resin in free base form; and wherein the weak basic anion exchange resin is of the gel type.

39.-40. (canceled)
